(19) United States
(12) Patent Application Publication
(10) Pub. No.: US 2003/0153744 A1
(43) Pub. Date: Aug. 14, 2003

(54) Anti-viral 7-deaza L-nucleosides

(75) Inventors: Khalid Mekouar, Laval (CA); Robert Deziel, Montreal (CA); Samir Mounir, Laval (CA); Radhakrishnan P. Iyer, Shrewsbury, MA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Micrologix Biotech Inc., Vancouver (CA)

(21) Appl. No.: 10/326,573

(22) Filed: Dec. 20, 2002

Related U.S. Application Data
(60) Provisional application No. 60/342,792, filed on Dec. 21, 2001.

(51) Int. Cl. A61K 31/7076; A61K 31/7052; C07H 19/044; C07H 19/056

(52) U.S. Cl. 536/27.2; 536/28.6; 514/43

(57) ABSTRACT

The present invention comprises 7-deaza L-nucleosides having unexpectedly high inhibitory activity against the hepatitis B virus. The invention further comprises pharmaceutical compositions comprising such compounds as well as methods of treating mammals, particularly humans, infected with HBV and other viral infections.

### BACKGROUND OF THE INVENTION

1. Field of the Invention

The present invention is in the field of anti-viral agents, particularly anti-viral L-nucleosides, and more particularly anti-viral 7-deaza L-nucleosides.

2. Description of the Related Art

Nucleoside and nucleotide analogs have long been studied as potential antiviral compounds. A number of D-nuceloside analogs are presently used as antiviral agents, including HIV reverse transcriptase inhibitors (such as AZT, ddI, ddC, and d4T). Similarly, purine D-nucleoside analogs have also been explored in search of immunomodulators.

Guanosine analogs having substituents at the 7- and/or 8-positions, for example, have been shown to stimulate the immune system (for a review, see Weigle, W. O., CRC Crit Rev. Immunol. 7:285,1987; Lin et al., J. Med. Chem. 28:1194, 1985; Reitz et al., J. Med. Chem. 37:3561, 1994; Michael et al., J. Med. Chem. 36:3431,1993). 7-Deazaguanosine and analogs have been shown to exhibit antiviral activity in mice against a variety of RNA viruses, even though the compound lacks antiviral properties in cell culture. 3-Deazaguanine nucleosides and nucleotides have also demonstrated significant broad spectrum antiviral activity against certain DNA and RNA viruses (Revanker et al., J. Med. Chem. 27:1389,1984). Certain 7- and 9-deazaguanine L-nucleosides exhibit the ability to protect mice against lethal challenge of Semliki Forest virus (Girgis et al., J. Med. Chem. 33:2750, 1990) (see also WO 98/16184, which discloses purine L-nuceloside analogs as antiviral agents).

Certain 6-sulfenamide and 6-sulfinamide purine nucleosides have demonstrated anti-tumor activity (Robins et al., U.S. Pat. No. 4,328,336). Certain pyrimido[5,4-D]pyrimidine nucleosides were effective in the treatment against L1210 in BDF1 mice (Robins et al., U.S. Pat. No. 5,041,542), and there, the antiviral and anti-tumor activities of the above mentioned nucleosides were suggested to be the result of their role as immunomodulators (Bonnet et al., J. Med. Chem. 36:635,1993).

Despite all the investigation, at present, there are no specific treatments for benign acute viral hepatitis. Use of adrenocorticosteroids, recommended by some, appears to have no effect curing the underlying disease. Furthermore, it appears that use of steroids in early treatment of hepatitis B virus (HBV) infection may result in the development of a persistent infection. Therapeutic effectiveness of interferon use on the prognosis and course of acute HBV infection remain unknown.

A number of strategies have been used in the treatment of chronic HBV, wherein the goals of treatment are three-fold: (1) to eliminate infectivity and transmission of HBV to others, (2) to arrest the progression of liver disease and improve the clinical prognosis, and (3) to prevent the development of hepatocellular carcinoma (HCC). Currently, there are several treatments being used. Interferon-α use is most common, but now lamivudine (3TC), and others are being looked at as potential therapeutic agents. None of these treatments can be called a cure, so a true cure for HBV and associated disease still remains elusive.

Therefore, a need exists for identifying compounds having improved anti-viral activity that are not toxic and/or cause other undesirable side effects. The present invention meets such needs, and further provides other related advantages.

### BRIEF SUMMARY OF THE INVENTION

The present invention comprises 7-deaza L-nucleosides having unexpectedly high inhibitory activity against the hepatitis B virus. In one aspect, the invention comprises compounds of structure (I):

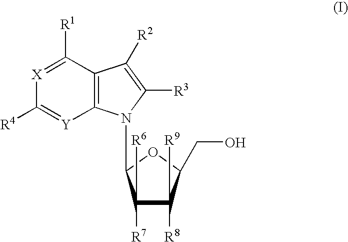

and pharmaceutically acceptable salts thereof, wherein

a) R¹ is H, C₁-C₆-alkyl, —Cl, —OH, C₁-C₄-alkoxy, —NH₂, or —NHZR⁵;

b) R² and R³ independently are —H, C₁-C₆-alkyl, methyl, C₂-C₆-alkenyl, C₂-C₆ alkynyl, —Cl, —I, —Br, —F, or heterocyclyl; or R² and R³together with the carbons to which they are attached form a 5 membered ring;

c) R⁴ is —NHZR⁵ or —N(R⁵)₂, wherein Z is —CO— or —SO₂ and R⁵ is C₁-C₆-alkyl, C₅-C₆ cycloalkyl, or aryl; or R⁴ is H, —OH, C₁-C₆-alkyl, C₁-C₆-alkenyl, C₁-C₄-alkoxy, or —NH₂;

d) X and Y are independently —N— or —CH—; and

e) R⁶, R⁷, R⁸, and R⁹ are independently —H, —OH, C₁-C₆-alkyl, —NH₂, —NHZR⁵, —F, —Cl, or —Br.

Compounds of the invention show unexpectedly high activity inhibiting hepatitis B virus replication. Accordingly, in another aspect, the invention comprises a method of inhibiting hepatitis B comprising administering to a mammal infected with hepatitis B an effective amount of a compound of the invention to slow or prevent hepatitis B replication.

### DETAILED DESCRIPTION OF THE INVENTION

The present invention is directed generally to anti-viral compounds, such as anti-hepatitis B virus (HBV) compounds. In one preferred embodiment, the present invention provides anti-viral compounds of structure (I):

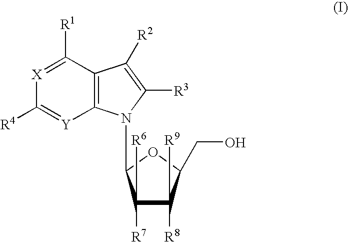

and pharmaceutically acceptable salts thereof, wherein

a) R¹ is H, C₁-C₆-alkyl, —Cl, —OH, C₁-C₄-alkoxy, —NH₂, or —NHZR⁵;

b) R² and R³ independently are —H, C₁-C₆-alkyl, methyl, C₂-C₆-alkenyl, C₂-C₆ alkynyl, —Cl, —I, —Br, —F, or heterocyclyl; or R² and R³together with the carbons to which they are attached form a 5 membered ring;

c) R⁴ is —NHZR⁵ or —N(R⁵)₂, wherein Z is —CO— or —SO₂ and R⁵ is C₁-C₆-alkyl, C₅-C₆ cycloalkyl, or aryl; or R⁴ is H, —OH, C₁-C₆-alkyl, C₁-C₆-alkenyl, C₁-C₄-alkoxy, or —NH₂;

d) X and Y are independently —N— or —CH—; and

e) R⁶, R⁷, R⁸, and R⁹ are independently —H, —OH, C₁-C₆-alkyl, —NH₂, —NHZR⁵, —F, —Cl, or —Br.

In certain preferred embodiments, the invention comprises compounds having structure (I), wherein:

a) R¹ is —NH₂, R² and R³ are independently —H, —F, methyl, or C₁-C₄-alkyl, and R⁴ is —H;

b) R¹ is —NH₂, R² is —H, R³ is —H, and R⁴ is —C₁-C₄-alkyl;

c) R¹ is —NHZR⁵;

d) R¹ is —NH₂, R² and R³ together with the carbons to which they are attached form a 5-membered ring, and R⁴ is —H;

e) R¹ is —H or C₁-C₄-alkyl, R² is —H R³ is —H, and R⁴ is H; or

f) R¹ is —NH₂, R² and R³ are —H or are independently —H or C₁-C₄-alkyl, and R⁴ is —NHZR⁵.

In another preferred embodiment, the present invention comprises compounds having structure (I), wherein:

a) R⁶ is —H, R⁷ is —H, and R⁸ is —OH, and R⁹ is —H;

b) R⁶ is —H, R⁷ is —OH, and R⁸ is —OH, and R⁹ is —H;

c) R⁶ is —H, R⁷ is C₁-C₄-alkyloxy, R⁸ is —OH, and R⁹ is —H;

d) R⁶ is —H, R⁷ is —NHZR⁵, R⁸ is —OH, and R⁹ is —H;

e) R⁶ is —H; R⁷ is —F, and R⁸ is —OH;

f) R⁶ is —OH or F, R⁷ is —H, and R⁸ is —H or —OH; or

g) R⁶, R⁷, and R⁸ are —H, and R⁹ is —OH or —F.

In still another preferred embodiment, the present invention comprises the compound of structure (II):

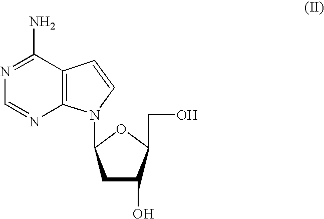

In another embodiment, compounds of the invention comprise those disclosed above in which the ribose moiety is an open chain (rather than a closed ring), wherein the bond between the oxygen and the 1′ carbon is omitted and the 1′ carbon is a methylene and the 4′ carbon bears a hydroxyl group.

As used herein, the term “heterocyclyl” refers to a C₅-C₁₀ mono- or bicyclic alkyl, alkenyl, or alkynyl moiety with a single free valence as defined above wherein one or more ring carbon atoms is replaced with a heteroatom (O, N, or S).

Compounds of the instant invention show surprising and exceptionally strong inhibition of HBV replication. Certain compounds of the invention, including L-7-deaza adenosine, exhibited antiviral activity against HBV with IC₅₀ in the range of 5 to 15 nM in an HBV cell-based assay. Accordingly, the compounds of the invention are useful research tools for in vitro and cell based assays to study the biological mechanisms of HBV infection, growth, and reproduction. The compounds of the invention are also useful for treating mammals, preferably humans, infected with HBV or other viral infections.

In another aspect, the invention comprises a pharmaceutical composition comprising any of the aforementioned compounds (or a pharmaceutically active salt or derivative thereof and a pharmaceutically acceptable carrier, diluent, or excipient. In one preferred embodiment, any of the aforementioned compositions are sterile.

In another aspect, the invention comprises a method of treating a mammal, preferably a human, with an effective amount of a composition as described herein.

As used herein, the term “pharmaceutically acceptable salts or complexes” refers to salts or complexes that retain the desired biological activity of the above-identified compounds and exhibit minimal or no undesired toxicological effects. Examples of such salts include, but are not limited to, acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesufonic acid, naphthalenedisulfonic acid, and polygalacturonic acid. The compounds may also be administered as pharmaceutically acceptable quaternary salts known by those skilled in the art, which specifically include the quaternary ammonium salt of the formula —NR+Z—, wherein R is hydrogen, alkyl, or benzyl, and Z is a counter ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

As used herein, the term “pharmaceutically active derivative” refers to any compound of the instant invention that upon administration to the subject in need thereof, is capable of providing directly or indirectly, the compounds with anti-viral activity as disclosed herein.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.01 to 300 mg/kg, preferably 0.1 to 100 mg/kg per day, and more preferably 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If a derivative exhibits activity similar to a parent compound, the effective dosage can be estimated as above using the weight of the derivative, or by other means known to those skilled in the art.

The methods of the invention comprise administration to a mammal (preferably human), suffering from a viral infection (e.g., HBV), a pharmaceutical composition according to the invention in an amount sufficient to alleviate the condition. The compound is conveniently administered in any suitable unit dosage form, including but not limited to one containing 1 to 3000 mg, preferably 5 to 500 mg of active ingredient per unit dosage form. A oral dosage of 1-500, preferably 10-250, more preferably 25-250 mg is usually convenient.

The active ingredient should be administered to achieve peak plasma concentrations of the active compound of about 0.001-30 μM, preferably about 0.01-10 μM. This may be achieved, for example, by oral administration or intravenous injection of a solution or formulation of the active ingredient, optionally in saline, or an aqueous medium or administered as a bolus of the active ingredient.

The concentration of active compound in the drug composition will depend on absorption, distribution, inactivation, and excretion rates of the drug, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials may be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it may contain, in addition to material of the above type, a liquid carrier such as a fatty oil. In addition, dosage unit forms can contain various other materials which modify the physical form of the dosage unit, for example, coatings of sugar, shellac, or enteric agents. See generally “Remington's Pharmaceutical Sciences,” Mack Publishing Co., Easton, Pa.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable derivatives or salts thereof can also be provided with other active materials that do not impair the desired action, or with materials that supplement the desired action, such as antibiotics, antifungals, other anti-inflammatories, or other antiviral compounds.

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS), and preferably the compositions are sterile.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (CA) and Gilford Pharmaceuticals (Baltimore, Md.). Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidylcholine, arachadoyl phosphafidylcholine; and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

The Examples provided below are merely illustrative and are not intended to be limiting. All patents, patent applications, and other publications are hereby incorporated by reference in their entirety.

### EXAMPLES

### EXAMPLE 1

### 4-AMINO-7-(2′-DEOXY,-β-L-ERYTHRO-PENTOFURANOSYL)PYRROLO[2,3-D]PYRIMIDINE (7-DEAZA-2′-DEOXY-L-ADENOSINE)

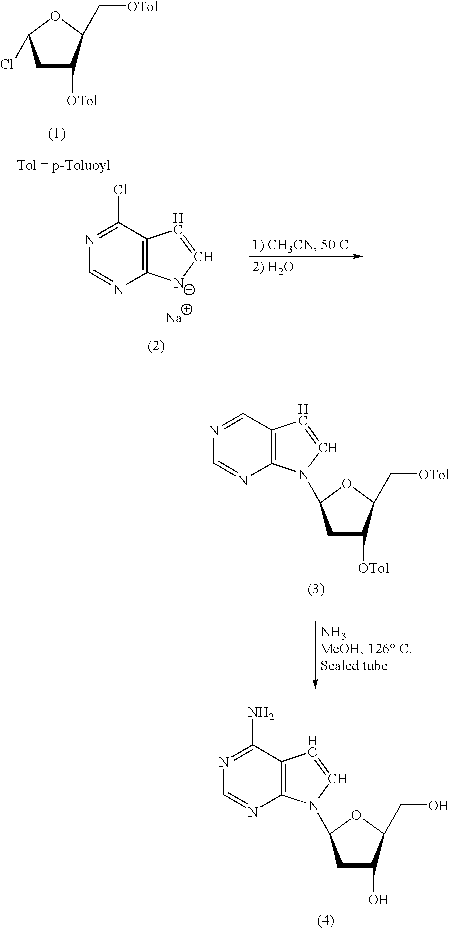

4-Chloro-7-(2′-deoxy-3′,5′-di-O-p-toluoyl-β-L-erythro-pentofuranosyl)pyrrolo[2,3-d]prrimidine (3)

To a suspension of the sodium salt of 4-chloropyrrolo [2,3-d] pyrimidine 2 (0.791 g, 5.15 mmol) in anhydrous CH₃CN (31 ml) was added sodium hydride 95% (0.14 g, 5.3 mmol) and the mixture was stirred at room temperature under argon atmosphere for 30 min. 1-chloro-2′-deoxy-3′,5′-di-O-p-toluoyl-α-L-erythro-pentofuranose 1(2 g; 5.15 mmol) was added portion-wise over a period of 30 min.

The reaction mixture was stirred at 50° C. for 2 hours, then at room temperature and filtered to remove insoluble material. After evaporation of the filtrate the residue was purified over a silica gel column using a gradient of ethylacetate-hexane (20%; then 25% ethylacetate, dry pack with silica gel/ethylacetate) to afford 1.25 g (68%) of 4-Chloro-7-(2′-deoxy-3′,5′di-O-p-toluoyl-β-L-erythro-pentofuranosyl)pyrrolo[2,3-d]pyrimidine 3.

MS (ES): m/z 506.2, [M+H]⁺

mp 115.9-117.9° C.

[α]_{D}+79.2 (c=0.48, CHCls)

¹H NMR (CDCl³): δ 2.44 (s, 3); 2.46 (s, 3); 2.76-2.86 (m, 1); 2.87-2.97 (m, 1); 4.58-4.78 (m, 3); 5.78 (m, 1); 6.62 (d, 1, J=3.59 Hz); 6.82 (t, 1, J=6.78 Hz); 7.25 (d, 2, J=8.57 Hz); 7.30. (d, 2H, J=7.50 Hz), 7.45(d, 1, J=3.58 Hz); 7.93 (d, 2, J=8.71 Hz), 7.99 (d, 2, J=7.32 Hz); 8.65 (s, 1).

¹³C NMR (CDCl₃) δ 166.1, 166.0, 152.3, 151.1, 150.8, 144.4, 144.1, 129.8, 129.6, (129.2 *2), 126.7, 126.4, 126.0, 118.3, 101.0, 84.4, 82.4, 75.0, 64.1, 38.1, 21.7, 21.6.

4-Amino-7-(2′-deoxyl-β-L-erythro-pentofuranosyl)pyrrolo2,3d]pyrimidine (4)

A solution of 3 (1.25 g; 2.47 mmol) in methanolic ammonia (saturated at 0° C., 30 ml) was heated in a sealed tube at 126° C. for 15 hours, then the mixture was evaporated to dryness. The residue was dissolved in water (60 ml) and washed with dichloromethane (4×30 ml). Evaporation of water under reduced pressure, followed with reverse phase purification (C-18) using as solvent: water-acetonitrile (gradient: 100%; 95%) afforded 0.4 g (65%) of 4-Amino-7-(2-deoxy-β-L-erythro-pentofuranosyl)pyrrolo[2,3-d]pyrimidine 4.

MS (ES): m/z 251.2, [M+H]⁺

mp 214.8-215.5° C.

[α]_{D}+39.3 (c=0.40, DMSO)

¹H NMR (DMSO): δ 2.10-2.17 (m, 1); 2.5 (m, 1); 3.46-3.58 (m, 2); 3.81 (m, 1); 4.33 (m, 1); 5.13 (t, OH, J=5.03 Hz); 5.23 (m, OH); 6.47 (t, 1, J=6.75 Hz); 6.57 (s, 1); 7.00 (s, NH₂); 7.33 (s, 1); 8.00 (s, 1).

¹³C NMR (DMSO-d6) δ 157.5, 151.6, 149.7, 121.7, 103.0, 99.7, 87.3, 83.4, 71.2, 62.2, 39.7.

References:

Reference 1: Kaimierczuk, et al., J. Am. Chem. Soc. 1984, 106 (21), 6379-6382.

Reference 2: a) Zhang, W.; Ramasamy, K. S.; Averett, D. R. Nucleosides & Nucleotides. 1999, 18(11&12), 2357-2365. b) Urata, H.; Ogura, E.; Shinohara, K.; Ueda, Y.; Akagi, M. Nucleic Acids Research. 1992, 20 (13), 3325-3332.

### Example 2

### Cell-Based Assays

Cell line

The HBV producing cells 2.2.15 are growth in RPMI 4% FBS, 5 mM L-glutamine (Bio Media), 0.75% sodium pyruvate (Bio Media). After six passages the cells are selected with 330 ug/ml of G418 during 10 days. All culture dishes used for the 2.2.15 cells are coated with a thin layer of rat tail collagen at 0.25 mg/ml diluted into 2 ml of sterile 0.2° acetic acid (Boehringer).

Antiviral assay

The 2.2.15 cells are plated at 1.6×10⁴ cells/wells in 96 well flat-bottomed plates. Cells are incubated 2 days in RPMI 4% FBS. The same procedure is followed for the treatment of cells used for cellular DNA analysis except that the cells are plated at 1×10⁵/well in 24 well flat-bottomed plates. The cells were treated with 9 consecutive daily doses of the compounds. The dry compounds are solubilized at 1 mM in sterile ddH₂O to constitute the working stock. In the case of the 3TC control, the original stock is diluted in 100% DMSO at 10 mM. A working stock solution at 100 μM is prepared in ddH₂O by dilution of the original stock. For the antiviral screening a serial dilution of the compounds is prepared in RPMI. 2% FBS. Freshly diluted compounds are added each day during 9 days. On day 10, the cells and the supernatants are collected for analysis.

Dot blot analysis of the extracellular HBV DNA

Cell supernatants are centrifuged at 2000 rpm for 10 minutes at 4° C. to eliminate any residual cells. The supernatant are then transferred to a new 96 well plate and treated with 0.2 mg/ml of protease at 56° C. for 1 hour. The supernatant are diluted with an equal volume of 2M NaOH/20×SSC buffer and incubated at least 30 minutes at room temperature. The samples are loaded on a nylon membrane using a dot blot apparatus (Bio-Rad). The membranes are washed with 0.5 ml of 1.0 M Tris-HCl (pH 7.4)/2 M NaCl followed by 0.5 ml 20×SSC. The membranes are dried and irradiated 6 minutes on the UV trans-illuminator. The membranes are then hybridized during 48 hours at 42° C. with a 1.2-kb HBV specific ³²P-labelled probe (Ready-To-Go labelling dCTP beads, Amersham). Membranes are washed for 15 minutes with 150 ml of 2×SSC, 0.1% (w/v) SDS at room temperature, 10 minutes with 150 ml of 1×SSC, 0.1% (w/v) at room temperature, 10 minutes with 150 ml of 1×SSC, 0.1% (w/v) SDS at 65° C. and finally 10 minutes with 150 ml of 0.1×SSC, 0.1% (w/V) SDS at 65° C.

Cellular toxicity evaluation

A panel of four cell lines, HepG2, NIH 3T3, Vero, HFF and human blood mononuclear cells are used for the evaluation of cell cytotoxicity profile of the compound using a non-radioactive tetra-zolium-based assay (MTT). The inhibition of cell proliferation is evaluated after a four days treatment of the cells with compounds in 96 well plates. The compounds are diluted in complete DMEM 2% FBS for the cell lines and in complete RPMI 10%FBS for the PBMC. On day 5, 15 μl of dye solution (Promega) containing tetrazolium salt are added to each well and incubated at 37° C. for 4 hours. A 100 μl of stop solution is added to solubilize the product of the reaction (formazan). The plates are incubated 4 hours at room temperature and read on the spectrophotometer at 570 nm.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of formula (I): 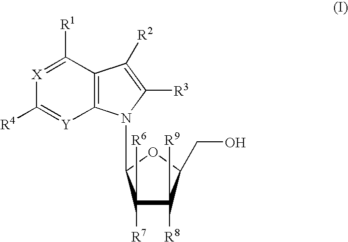
and pharmaceutically acceptable salts thereof, wherein
a) R¹ is H, C₁-C₆-alkyl, —Cl, —OH, C₁-C₄-alkoxy, —NH₂, or —NHZR⁵;
b) R² and R³ independently are —H, C₁-C₆-alkyl, methyl, C₂-C₆-alkenyl, C₂-C₆ alkynyl, —Cl, —I, —Br, —F, or heterocyclyl; or R² and R³ together with the carbons to which they are attached form a 5 membered ring;
c) R⁴ is —NHZR⁵ or —N(R⁵)₂, wherein Z is —CO— or —SO₂— and R⁵ is C₁-C₆-alkyl, C₅-C₆-cycloalkyl, or aryl; or R⁴ is H, —OH, C₁-C₆-alkyl, C₁-C₆-alkenyl, C₁-C₄-alkoxy, or —NH₂;
d) X and Y are independently —N— or —CH—; and
e) R⁶, R⁷, R⁸, and R⁹ are independently —H, —OH, C₁-C₆-alkyl, —NH₂, —NHZR⁵, —F, —Cl, or —Br.

2. The compound according to claim 1, wherein:
a) R¹ is —NH₂, R² and R³ are independently —H, methyl, —F, or C₁-C₄-alkyl, and R₄ is —H;
b) R₁ is —NH₂, R² is —H, R³ is —H, and R⁴ is —C₁-C₄-alkyl;
c) R¹ is —NHZR⁵;
d) R¹ is —NH₂, R² and R³ together with the carbons to which they are attached form a 5-membered ring, and R⁴ is —H;
e) R¹ is —H or C₁-C₄-alkyl, R² is —H R³ is —H, and R⁴ is H; or
f) R¹ is —NH₂, R² and R³ are —H are independently —H or C₁-C₄-alkyl, and R⁴ is —NHZR⁵.

3. The compound according to claim 1, wherein:
a) R⁶ is —H, R⁷ is —H, and R⁸ is —OH, and R⁹ is —H;
b) R⁶ is —H, R⁷ is —OH, and R⁸ is —OH, and R⁹ is —H;
c) R⁶ is —H, R⁷ is C₁-C₄-alkyloxy, R⁸ is —OH, and R⁹ is —H;
d) R⁶ is —H, R⁷ is —NHZR⁵, R⁸ is —OH, and R⁹ is —H;
e) R⁶ is —H, R⁷ is —F, and R⁸ is —OH;
f) R⁶ is —OH or F, R⁷ is —H, and R⁸ is —H or —OH; or
g) R⁶, R⁷, and R⁸ are —H, and R⁹ is —OH or —F.

4. The compound according to claim 1 having structure (II):

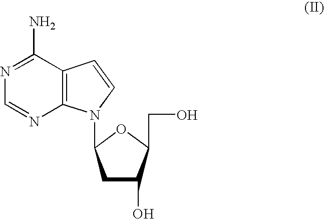

5. A pharmaceutical composition comprising a compound according to any one of claims 1-4 and a pharmaceutically acceptable carrier.

6. A method of treating a mammal infected with HBV, the method comprising administering to the mammal an effective amount of a composition according to claim 5.

7. The method according to claim 6, wherein the mammal is a human.
